(19) United States
(12) Patent Application Publication
(10) Pub. No.: US 2010/0184022 A1
(43) Pub. Date: Jul. 22, 2010

(54) Hybridization Probe Assay and Array

(76) Inventor: Brigitte Desiree Alberte Colau, Rixensart (BE)

Correspondence Address: GlaxoSmithKline;GLOBAL PATENTS -US, UW2220, P. O. BOX 1539, KING OF PRUSSIA, PA, 19406-0939, US

(21) Appl. No.: 12/161,179

(22) PCT Filed: Jan. 16, 2007
(86) PCT No.: PCT/EP2007/050380

§ 371 Date: Mar. 29, 2010

(30) Foreign Application Priority Data
Jan. 17, 2006 (GB) 0600927.8

(51) Int. Cl. C12Q 1/68; C07H 21/04

(52) U.S. Cl. 435/6; 536/24.3; 536/24.2

(57) ABSTRACT

A probe suitable for coupling with a particulate support such as a microbead, the probe comprising:
a) a coupling group which permits coupling of the probe to the surface of the particulate support;
b) a spacer; and
c) a target-specific oligonucleotide probe sequence,
   wherein the spacer comprises:
i) an oligonucleotide spacer of at least 15 nucleotides between the target specific probe sequence and the support coupling group; and optionally
ii) a carbon spacer of between 3 and 50 carbon units between the target specific probe sequence and the support coupling group.

The present invention relates to analysis of interactions between molecules.

### BACKGROUND OF THE INVENTION

Single nucleotide polymorphisms (SNPs) are recognized as an important cause of variety in biological function. Although SNPs can have important effect, most genetic variety is observed in the non-coding DNA sequences. Besides the importance of SNPs in human genetics, SNP detection is also important in the field of infectious diseases. For human papilloma virus (HPV) infections genotyping is an important indicator. Nowadays, the family of HPVs comprises more than one hundred genotypes, which can be classified in different groups including important human pathogens (de Villiers et al, 2004). In particular the high-risk HPV types are known to induce cervical cancer. Therefore, recognition of these high-risk types requires a robust tool for diagnosis enabling the most adequate treatment.

Nucleic acid assays are based on the detection of specific DNA or RNA sequences. Target nucleic acids, e.g. derived from clinical samples, can be recognized by labeled detection probes. The specificity of the assay is determined by the specificity of the hybridization process between target and probe. Detection of SNPs however, requires the highest level of specificity. In addition, at present many techniques are available to detect SNPs (e.g. hybridization, sequencing, and mass spec analysis), but none of them efficiently combines high throughput and high density screening of SNPs. Nevertheless, the need is growing for such a tool.

The use of beads (or microbeads), such as spherical beads also referred to herein as microspheres, in multiplex analysis has been described previously in, for example, Dunbar S A. (Applications of Luminex® xMAPtrade mark technology for rapid, high-throughput multiplexed nucleic acid detection. Clin Chim Acta. 2005 Aug. 15); [Epub ahead of print], Clin Chim Acta. 2006 January; 363(1-2):71-82., see http://www.ncbi.nlm.nih.gov/entrez/query.fcgi?cmd=Retrieve&db=pubmed&dopt=Abstract&list_uids=16102740&query_hl=1) and through the Luminex™ product information and website (www.Luminex™corp.com). The Luminex™ system is a bead-based multiplexing (array) technology which has proven to be very powerful for analyzing multiple parameters or analytes within one sample (Dunbar et al, 2005). It delivers results on many bio assay formats including nucleic acid assays, receptor-ligand assays, immunoassays and enzymatic assays.

The use of liquid bead microarrays for HPV detection is discussed in Wallace J et al, (Facile, comprehensive, high-throughput genotyping of human genital papillomaviruses using spectrally addressable liquid bead microarrays.” J Mol Diagn. 2005 February; 7(1):72-80.)

Whilst protocols and materials are known and published for Luminex™ type systems, and are given on the Luminex website, there is still a need to improve upon such techniques and materials. For example, we have found that some standard protocols for the use of liquid bead microarrays are not effective for different SNPs in small targets, or for systems where there are multiple point mutations which are not always in the middle of the probe. The TMAC system generally used by Luminex also recommends a constant probe length within a given multiplex reaction. Moreover, TMAC is toxic and unstable at higher temperatures:

The present invention addresses such a need for improvements in probe and protocol design suitable for use with bead based analysis systems such as Luminex.

### STATEMENTS OF INVENTION

The present invention relates to a method for the detection of any interaction between a probe and a target nucleic acid, the method comprising the steps of:
i Denaturation of any double stranded target polynucleic acid present in a sample;
ii Hybridisation of the denatured target with probe under conditions that allow specific hybridization between probe and target to occur;
iii Optionally, stringent washing;
iv Addition of, and incubation with, reporter molecule to allow detection of probe-target binding;
v Optionally, washing; and
vi Detection of probe-target binding,
   wherein the method comprises one of more of the following additional steps:
a maintenance of the hybridization temperature after the hybridization step between probe and target after step (ii);
b use of a dilution-wash step immediately after hybridization step (ii);
c Maintenance of the hybridization temperature during any stringent wash at step (iii);
d Shaking or mixing with heating at step (ii) e.g. by use of a thermo-mixer at step (ii);
e Maintenance of the hybridization temperature during incubation with the reporter molecule at step (iv);

In one aspect the hybridization temperature is maintained from step (ii) until the reaction with a reporter molecule is complete in step (iv).

In one aspect steps a and c are performed, that is the hybridization temperature is maintained after the hybridization step between probe and target and during a stringent washing step at step (iii).

In a further aspect the probe is coupled to a particulate support such as a bead.

The invention also relates to a probe suitable for coupling with a particulate support such as a bead, the probe comprising:
a) a coupling group (such as an NH₂ group) which permits coupling (such as covalent coupling) of the probe to the surface of the particulate support;
b) a spacer; and
c) a target-specific oligonucleotide probe sequence,
   wherein the spacer comprises:
i) an oligonucleotide spacer of at least 15 nucleotides, such as a thymine repeat spacer or a spacer comprising a TTG repeating unit, between the target specific probe sequence and the coupling group; and optionally
ii) a carbon spacer of between 3 and 50 or between 13 and 50 carbon units, suitably a C₁₈ spacer, between the target specific probe sequence and the coupling group.

In one aspect the spacer is at the 3′ end of the target specific probe sequence.

In another aspect the spacer is at the 5′ end of the target specific probe sequence.

The invention also relates to a set of probes as described herein, comprising at least two different target specific probe sequences coupled to different particulate supports which are distinguishable from one another, for example by means of different labels such as fluorescent labels or barcodes.

The invention also relates to a set of from 2 to 1000 for example 2 to 50 different target specific probes, each probe comprising:
a) a coupling group which permits coupling of the probe to a solid support;
b) a spacer; and
c) a target-specific oligonucleotide probe sequence,
   wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group, which oligonucleotide spacer does not hybridise to the target or to a flanking region of the target.

The invention also relates to spacer sequences per se as defined in any aspect of the invention herein.

The invention also relates to kits comprising a spacer molecule of the invention and a particulate support such as a bead.

The invention also relates to a kit comprising a spacer molecule of the invention and instructions for coupling to a particulate support such as a bead.

The invention also relates to a particulate support such as a bead coupled to a probe as defined herein.

The invention also relates to a kit comprising a particulate support such as a bead coupled to a spacer molecule of the invention and instructions for use in detection of a target molecule.

The invention also relates to a kit comprising a particulate support such as a bead coupled to a probe of the invention and instructions for use in detection of a target molecule.

The invention also relates to a kit comprising a probe which probe comprises:
a) a coupling group which permits coupling of the probe to a surface of a particulate support;
b) a spacer; and
c) a target-specific oligonucleotide probe sequence,
   wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group;
   and a particulate support such as polystyrene beads.

The invention also relates to a kit comprising a probe which probe comprises:
a) a coupling group which permits coupling of the probe to a surface of a particulate support;
b) a spacer; and
c) a target-specific oligonucleotide probe sequence,
   wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group;
   and instructions for coupling to a particulate support such as polystyrene beads.

### FIGURES

FIGS. 1a and 1b provide a general schematic overview of the probe & spacer design. FIG. 1c further develops this.

FIG. 2 provides an overview of an assay protocol for a bead based detection system.

### DETAILED DESCRIPTION

In the present invention improvements have been made to the protocols and reagents used in standard suspension bead assay detection of nucleic acids, such as the Luminex™ based assay.

Particulate supports for use in the present invention include in particular beads, which includes for example spherical beads or cylindrical beads. Beads may also be referred to as microbeads, or beads for use in microarrays. The description of the invention in relation to beads also applies to other particulate supports for use in the invention.

Beads for use in the present invention, and which includes microspheres described herein, are suitably beads that are suitable for use in flow cytometric analysis. Beads are suitably able to be coupled to a probe to detect interaction between a probe and a target. In one aspect beads are labelled with a unique fluorescent molecule or combination of molecules. Suitably the label on or in the beads is able to be identified by use of laser excitation of one or more fluorochromes within the bead. In one aspect the bead is a polystyrene bead. In another aspect the bead is a glass bead.

For example, the Luminex xMAP system incorporates 5.6 μm polystyrene microspheres that are internally dyed with two spectrally distinct fluorochromes (see Dunbar et al supra). Such beads are suitable for use in the present invention.

Other bead labelling systems for use in the invention include barcodes or digital holographic elements, for example barcode labelled cylindrical beads of Illumina Inc. Barcodes or digital holographic elements can be used as an alternative to fluorescent labels.

For example the Illumina VeraCode system incorporates cylindrical glass microbeads measuring 240 μm in length by 28 μm in diameter that have embedded into them digital holographic elements to create unique bead types. When excited by a laser, each VeraCode bead emits a unique code image which can be specifically detected.

In one aspect of the invention the beads may also have magnetic or paramagnetic properties.

Generally the beads are suitable for use in a multiplex system to detect simultaneously any interaction between multiple possible targets and multiple probes.

The examples herein use human papillomavirus (HPV) targets and probes but the principles developed can in principle be applied to detection of polynucleic acid from any source.

Standard molecular biology techniques are described in Sambrook et al, (Molecular cloning a laboratory manual, Cold Spring Harbour Press, third edition). Details of, for example, the principles and parameters relevant for hybridisation between probes and target, and the amplification of target polynucleic acid are described in EP1012348, incorporated herein by reference.

### Probes

The probe generally comprises (1) a coupling group (such as an NH₂ group), which permits (suitably covalent) coupling of the probe to the bead surface, (2) a spacer which serves to create a distance between the bead surface and the specific probe sequence, and (3) a target-specific oligonucleotide probe sequence (which may also be referred to herein as a target specific probe sequence).

In one aspect probes have a primary amino group suitable for coupling to a carboxyl group on a bead or other support.

In one aspect the invention relates to probes which contain target specific HPV probe sequences such as the published SPF10 probe sets (see EP1012348, incorporated herein by reference), by way of example for HPV, or any probe or combination of probes described herein, in particular those in Example 13, optionally linked with a polycarbon repeat.

In one aspect the invention is suitable for identification of SNPs occurring within a short fragment of target nucleic acid, generally a fragment of DNA amplified from a sample, such as a fragment of 20-50 bases, such as 20-30 bases, such as 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length.

In another aspect the invention is capable of discriminating between mismatches which are located at positions other than the middle of the probe. In one aspect the invention may be used to discriminate between mismatches which are 1, 2, 3, 4, 5, 6, 7, 8, 9, 10 or even further from the centre of the probe. In one aspect the invention may be used to discriminate between mismatches which are 1, 2, 3, 4, 5, 6, 7, 8, 9, 10 bases or even further from either end of the probe, suitably 3-10 bases.

The probes of the present invention allow discrimination of target from non target at sites close to the end of the probe which allows short target fragments to be probed for the presence of multiple different SNPs.

Carbon Based Spacers and Combinations with Oligo Spacers

In one aspect of the invention the probe comprises a carbon spacer of between 3 and 50 or between 13 and 50 carbon units, in one aspect a C20-C50 spacer, such as a C20-C40 spacer, or such as a C20-C30 spacer, between the target specific probe sequence and the coupling group. Any suitable spacer may be used, such as a C13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or C50 spacer. An appropriate spacer can be selected using standard techniques for an effect on the specificity of binding and signal intensity to obtain an optimum result.

In one aspect of the invention the probe comprises an oligonucleotide spacer, additional to that of the carbon spacer, the oligonucleotide spacer being at least 15 nucleotides or at least 20 nucleotides, such as from 15-150 or from 20-150 nucleotides, for example 25-100 nucleotides, 30-75 nucleotides, including 15-20, 20-25, 25-30, 30-35, 35-40, 40-45 and 45-50 nucleotides. The oligonucleotide spacer may be for example a homopolymer or a heteropolymer. In one aspect the oligonucleotide spacer is a poly thymine (poly T) spacer, or a spacer comprising other suitable repeating nucleotide units such as a (TTG) repeating spacer, or a poly A (adenine) spacer, or a poly G spacer, or a poly C spacer. Other heteropolymer spacers which may be suitable include repeats of TTTG, AAG, AAC, AAAG or AAAC. Different spacers may be tested to optimize probe-target interactions using routine methods well known in the art.

In one aspect the invention thus generally provides a probe comprising both a carbon spacer and an oligonucleotide spacer.

In one aspect the oligonucleotide spacer is located between a carbon spacer and a specific probe sequence. In such a case the carbon spacer may be shorter than 13 carbon units long, such as C12, or even shorter.

In one aspect, the oligonucleotide spacer is selected such that it does not hybridise to the target sequence or a flanking region of the target sequence. In one aspect the oligonucleotide spacer is selected such that it does not hybridise to the target sequence or to a flanking region of the target sequence when in use in the method described herein.

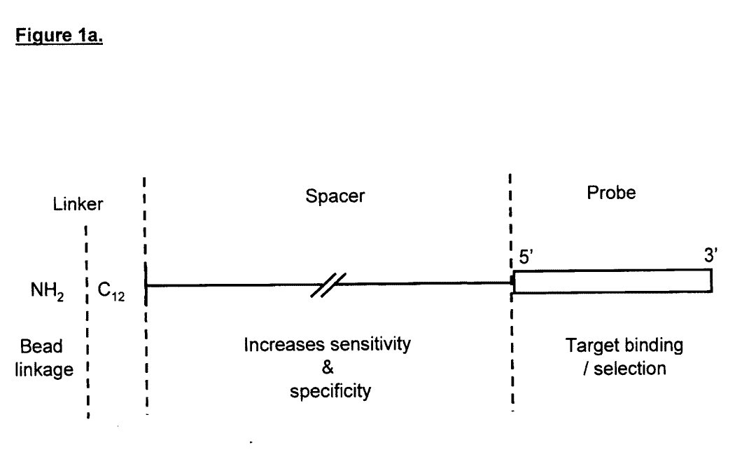

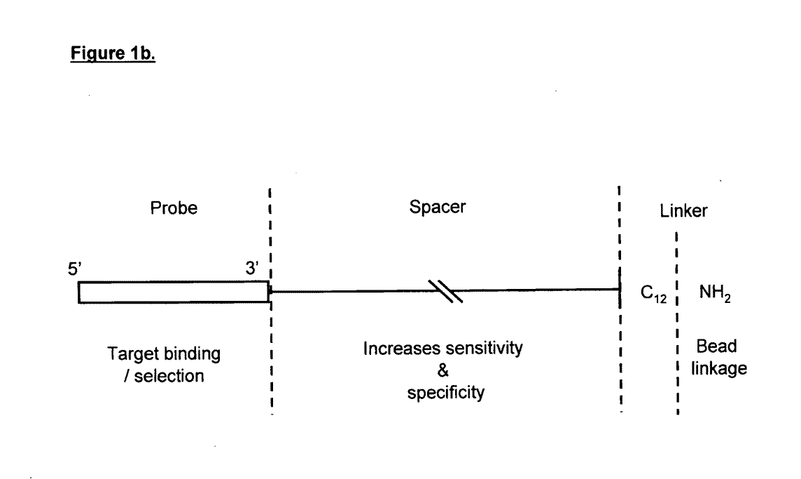

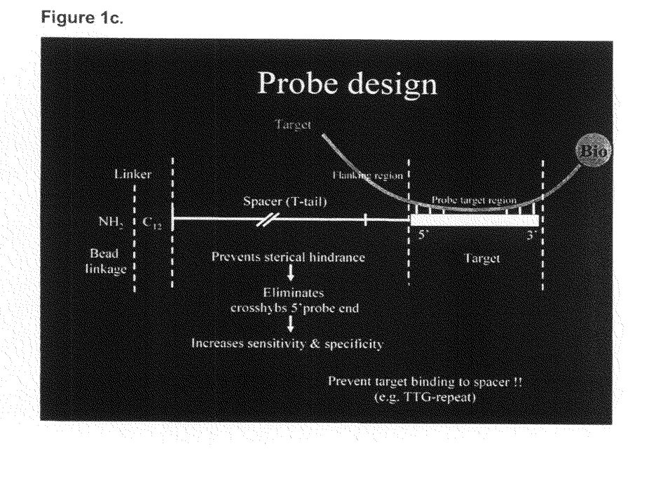

In one aspect the oligonucleotide spacer is selected such that the region of the spacer which flanks the target specific probe does not hybridise to the target sequence or to a flanking region of the target sequence. This is illustrated in FIG. 1c.

Poly carbon spacers are disclosed in Cowan et al (Transfer of a Mycobacterium tuberculosis genotyping method, Spoligotyping, from a reverse line-blot hybridization, membrane-based assay to the Luminex multianalyte profiling system. J Clin Microbiol. 2004 January; 42(1):474-7.) and Taylor et al (Taylor J D, Briley D, Nguyen Q, Long K, Iannone M A, Li M S, Ye F, Afshari A, Lai E, Wagner M, Chen J, Weiner M P. Flow cytometric platform for high-throughput single nucleotide polymorphism analysis. Biotechniques. 2001 March; 30(3):661-6, 668-9)

Carbon spacers are suitably (CH2)n spacers.

### Oligo Spacers

In one aspect the invention thus provides a probe comprising only an oligonucleotide spacer between the bead coupling group and a target-specific probe sequence (i.e. in the absence of a carbon spacer).

In one aspect this spacer is at least 15 nucleotides or at least 20 nucleotides. In one aspect this spacer is from 15-150 or from 20-150 nucleotides, for example 25-100 nucleotides, 30-75 nucleotides, including 15-20, 20-25, 25-30, 30-35, 35-40, 40-45 and 45-50 nucleotides.

The oligonucleotide spacer may be for example a homopolymer or a heteropolymer. In one aspect the oligonucleotide spacer is a poly thymine (poly T) spacer, or a spacer comprising other suitable repeating nucleotide units such as a (TTG) repeating spacer, or a poly A (adenine) spacer, or a poly G spacer, or a poly C spacer. Other heteropolymer spacers which may be suitable include repeats of TTTG, AAG, AAC, AAAG or AAAC. Different spacers may be tested to optimize probe-target interactions using routine methods well known in the art.

In one aspect the invention thus provides a probe comprising an oligonucleotide spacer between a bead coupling group and a target-specific probe sequence, wherein the oligonucleotide spacer is a polythymine (poly T) spacer.

In one aspect the invention thus provides a probe comprising an oligonucleotide spacer between the bead coupling group and a target-specific probe sequence, wherein the oligonucleotide spacer is a TTG repeat spacer or a polyA spacer.

In one aspect the spacer, (either a carbon+oligonucleotide spacer, or oligonucleotide spacer alone), is at the 3′ end of the target specific probe sequence. In another aspect the spacer is at the 5′ end of the target specific probe sequence.

In one aspect, the oligonucleotide spacer is selected such that it does not hybridise to the target sequence or a flanking region of the target sequence. In one aspect the oligonucleotide spacer is selected such that it does not hybridise to the target sequence or to a flanking region of the target sequence when in use in the method described herein.

In one aspect the oligonucleotide spacer is selected such that the region of the spacer which flanks the target specific probe does not hybridise to the target sequence or to a flanking region of the target sequence. This is illustrated in FIG. 1c.

In a further aspect the invention relates to a probe set comprising at least 2 probes, suitably including any probe or probes of the present invention, wherein at least one probe is linked to a bead or the spacer through the 5′ end of the probe, and wherein at least one probe is linked to a bead or the spacer through the 3′ end of the probe.

### Spacer Sequences

The invention also relates to spacer sequences suitable for use with liquid, bead based detection systems. Spacers according to the invention may be any spacers described herein. Spacers may comprise or consist of, for example, a poly carbon repeat (eg C₁₂-C₃₀) and an oligonucleotide repeat (eg polyT or poly (TTG) or polyA, of between 15-150 or 20-150 nucleotides in length) coupled together, and suitable for attachment to a target specific probe sequence.

Spacers of the invention may also comprise or consist of an oligonucleotide repeat of 15-150 or 20-150 or 25-150 nucleotides in total length.

Spacers suitably comprise a coupling group, such as a primary amino group, suitable for attachment to a bead.

The present invention also relates to a spacer molecule of the invention coupled to a bead.

The invention also relates to a spacer molecule of the invention coupled to a target specific probe sequence, and optionally also coupled to a bead.

### Kits

The invention also relates to a kit comprising a spacer molecule of the invention and a particulate support such as a bead.

The invention also relates to a kit comprising a spacer molecule of the invention and instructions for coupling to a particulate support such as a bead.

The invention also relates to a kit comprising a spacer molecule of the invention coupled to a particulate support such as a bead, with instructions for coupling to a target specific probe sequence.

The invention also relates to a kit comprising a probe of the present invention coupled to a particulate support such as a bead and instructions for use in detection of a target.

### Process

The present invention also relates to certain process improvements made to existing protocols for detecting probe-target interactions at the nucleic acid level using bead-based-technologies.

Bead based technologies such as the Luminex technology are well described in the art and literature. Beads, also referred to as microspheres, are suitably polystyrene beads as described in Dunbar et al, and references therein, all hereby incorporated by reference.

The general method of the invention is a standard scheme for the detection of any interaction between a probe, suitably a probe as defined herein, and a target nucleic acid. The method suitably comprises the steps of:
i Denaturation of any target polynucleic acid present in a sample;
ii Hybridisation of target with probe under conditions that allow specific hybridization between probe and target to occur;
iii Optionally, stringent washing to remove substantially all unbound materials
iv Addition of, and incubation with, reporter molecule to allow detection of probe-target binding;
v Optionally, washing; and
vi Detection of the probe-target binding.

A detailed example of such a protocol is given in the examples contained herein.

The general steps are suitably consecutive, but in one aspect certain steps may be performed together, for example, the probe being reacted simultaneously with target and reporter molecule.

Specific hybridization of a probe to a target nucleic acid generally means that said probe forms a duplex with part of this target region or with the entire target region under the experimental conditions used, and that under those conditions said probe does not form a duplex with other regions of the polynucleic acids present in a sample being analysed.

Stringent washing conditions are well known in the art and include for example 3×SSC, 0.1% Sarkosyl at 50° C., and those conditions described in the examples herein.

Washing at step (v) is carried out under any suitable conditions, well known in the art, to allow removal of excess reporter molecule, for example. In one aspect of the invention washing is carried out in the presence of a lower concentration of SSC than used in the washing step, such as substantially 2×SSC, 1.5×SSC, or substantially 1×SSC.

Detection may be carried out by any suitable method, with one aspect of the invention using flow cytometric analysis to detect target probe interaction based upon the fluorescent properties of beads such as the Luminex bead system described in Dunbar (supra). In particular, this paper indicates that, for example, the Luminex xMAP system incorporates 5.6 μm polystyrene microspheres that are internally dyed with two spectrally distinct fluorochromes. Using precise amounts of each of these fluorochromes, an array is created consisting of different microsphere sets with specific spectral addresses. Each microsphere set can possess a different reactant on its surface. Because microsphere sets can be distinguished by their spectral addresses, they can be combined, allowing e.g., 100 or more different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex® 100™ analyzer. A 635-nm 10-mW red diode laser excites the two fluorochromes contained within the microspheres and a 532-nm, 13-mW yttrium aluminum garnet (YAG) laser excites the reporter fluorochrome (R-phycoerythrin, Alexa 532, or Cy3) bound to the microsphere surface. High-speed digital signal processing classifies the microsphere based on its spectral address and quantifies the reaction on the surface. Thousands of microspheres are interrogated per second resulting in an analysis system capable of analyzing and reporting for example 100 or more different reactions in a single reaction vessel in just a few seconds per sample.

In on aspect of the invention the beads may be paramagnetic beads. In one aspect the beads may be mixed with the target and/or reporter using mechanical mixing based upon the magnetic properties of the beads.

In one aspect the method of the invention comprises maintenance of the hybridization temperature after the hybridization step between probe and target after step (ii). In one aspect there is maintenance of the hybridization temperature until at least the stringent wash at step (iii). In one aspect the method of the invention comprises maintenance of the hybridization temperature during incubation with the reporter molecule at step (iv).

As such, the present invention relates to a process as outlined above for the detection of any interaction between a probe and a target nucleic acid, wherein the temperature of the hybridization reaction between target and probe is maintained until the reaction with a reporter molecule is substantially complete.

In one aspect the method of the invention also comprises use of a dilution-wash step immediately after hybridization step (ii). Such a step increases the volume of the reaction between the target and probe, and appears to reduce the possibility for aspecific hybridization. Dilution may be carried out using the wash buffer used to remove any unbound materials in step ii of the method.

In one aspect the method of the invention comprises shaking or mixing while heating for example by use of a thermo-mixer at step (ii). A thermo-mixer is generally any device that provides mixing of a sample at a temperature that may be predetermined. Here the mixing is suitably at the hybridization temperature, generally 50° C. or higher such as between 50-55° C., such as 50° C., 52° C., 54° C. and 55° C.

In one aspect the method of the invention comprises washing of the final probe-target complex in 1×SSC before detection of signal after step (vi). Such washing may be carried out at room temperature.

In one aspect the method of the invention comprises shaking of the final probe-target complex before detection of signal after step (vi).

In a further aspect the method comprises the further step of coupling a bead with a probe before step (i). In this way the reaction between the target and probe takes place in the context of a solid support.

In a further aspect the invention relates to a method as outlined about wherein the probe is linked to a bead, suitably a polystyrene bead having a fluorochrome.

In a further aspect the invention relates to a method as outlined above wherein at least 2 probes are used simultaneously to detect different targets. Such reactions are generally referred to as Multiplex reactions. In one aspect probes of the present invention having different target specificity are attached to beads, each bead being specific for each type specific probe.

In a further aspect the invention relates to a multiplex reaction comprising at least 2 type specific probes, wherein the probes are attached to beads, suitably beads labeled with distinct fluorochromes, and wherein the probe length of different probes within the multiplex reaction is not identical. For example, where a defined polynucleotide (eg DNA) fragment will be simultaneously probed with multiple different type specific probes, then in one aspect the present invention does not require that all probes be of equal length, and in one aspect probes do differ in length.

In a further aspect the hybridization between probe and target is carried out in the presence of sodium citrate (SSC) or equivalent, such as from 2× to 4×SSC or 3×SSC, suitably to provide an ionic environment for probe-target interactions to occur.

The present invention is illustrated with respect to the following examples which are not limiting upon the invention.

### Materials & Methods:

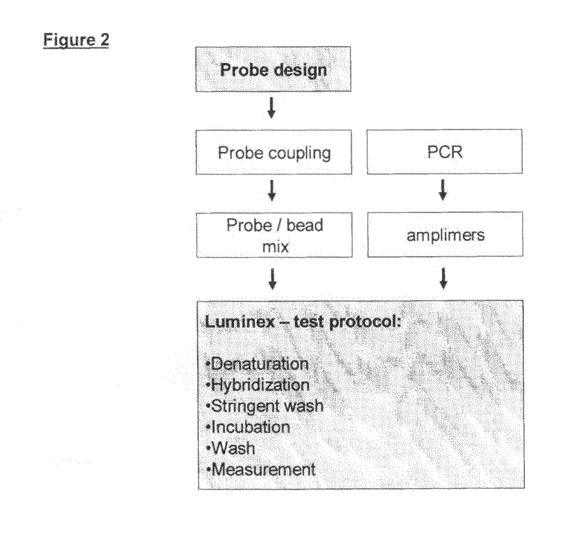

Standard hybridization procedure (step-wise) according to Wallace et al (2005) supra is as follows:
1. Select the appropriate oligonucleotide-coupled microsphere sets.
2. Resuspend the microspheres by vortex and sonication for approximately 20 seconds.
3. Prepare a Working Microsphere Mixture by diluting coupled microsphere stocks to 150 microspheres of each set/μl in 1.5×TMAC (1×TMAC=2 mol/l TMAC/0.15% Sarkosyl/75 mmol/l Tris, 6 mmol/l EDTA) Hybridization Buffer (Note: 33 μl of Working Microsphere Mixture is required for each reaction)
4. Mix the Working Microsphere Mixture by vortex and sonication for approximately 20 seconds.
5. To each sample or background well, add 33 μl of Working Microsphere Mixture.
6. To each background well, add 17 μl dH₂O.
7. To each sample well add amplified biotinylated DNA and dH₂O to a total volume of 17 μl (Note: 7 μl of a PCR reaction is used for detection).
8. Mix reaction wells gently by pipetting up and down several times.
9. Incubate at 99° C. for 5 minutes to denature the amplified biotinylated DNA in a thermocycler.
10. Incubate the reaction plate at hybridization temperature (55° C.) for 15 minutes.
11. During incubation, prepare a filter plate by rinsing twice with ice cold 1×TMAC. Next, fill each well of the filter plate with ice cold 1×TMAC.
12. During incubation, prepare fresh reporter mix by diluting streptavidin-R-phycoerythrin to 2 μg/ml in 1×TMAC hybridization buffer (Note: 75 μl of reporter mix is required for each reaction), and place it in an oven or water bath at the hybridization temperature.
13. Terminate the hybridization reaction by transferring the entire reaction to the filter plate containing ice cold wash buffer.
14. After transfer, wash the filter plate stringently twice with ice cold 1×TMAC wash buffer by intervening vacuum filtration.
15. Add 75 μl of reporter mix to each well and mix gently by pipetting up and down several times.
16. The entire plate is allowed to reach room temperature for approximately 30 minutes.
17. Incubate the reaction plate at hybridization temperature for 30 minutes.
18. Terminate the incubation by vacuum filtration.
19. Wash twice with 1×TMAC wash buffer by intervening vacuum filtration.
20. Dissolve a reaction in with 1×TMAC wash buffer by intervening vacuum filtration.
21. Analyze at room temperature on the Luminex™ 100 analyzer according to the system manual.

[See FIG. 2. General schematic overview of the work-flow as described by Wallace et al (2005)]

The sensitivity and specificity of the test is based on specific hybridization between probe and target nucleic acid sequences. Therefore, the hybridization and wash but also the incubation with PE appeared to be crucial steps in the procedure. The protocol was adapted in order to maximize the specificity and sensitivity of the reaction, by optimizing different parameters, such as temperatures and diffusion kinetics. These adaptations are indicated in the optimized hybridization protocol (see below).

### Materials:

### A. Buffers

0.1 M MES pH 4.5 (COUPLING BUFFER)

| | | Final | Amount/ |
|---|---|---|---|
| Reagent | Catalog Number | Concentration | 250 ml |
| MES (2[N- | Sigma M-2933 | 0.1 M | 4.88 g |
| Morpholino] | | | |
| ethanesulfonic acid) | | | |
| dH₂O | — | — | Up to 250 ml |
| 5 N NaOH | Fisher SS256-500 | — | ~5 drops |

Filter (45 μm) Sterilize and store at 4° C.

0.02% TWEEN (WASH BUFFER I)

| | | Final | Amount/ |
|---|---|---|---|
| Reagent | Catalog Number | Concentration | 250 ml |
| TWEEN 20 | Sigma P-9416 | 0.02% | 50 μl |
| (Polyoxyethylenesorbitan | | | |
| monolaurate) | | | |
| dH₂O | — | — | 250 ml |

Filter (45 μm) Sterilize and store at Room Temperature

20% Sarkosyl

| | | Final | Amount/ |
|---|---|---|---|
| Reagent | Catalog Number | Concentration | 250 ml |
| Sarkosyl (N- | Sigma L-9150 | 20% | 50 g |
| Lauroylsarcosine) | | | |
| dH₂O | — | — | 250 ml (adjust to) |

Filter (45 μm) Sterilize and store at Room Temperature

TE pH 8.0 (SAMPLE DILUENT)

| | | Final | Amount/ |
|---|---|---|---|
| Reagent | Catalog Number | Concentration | 250 ml |
| Tris EDTA Buffer | Sigma T-9285 | 1 X | 2.5 ml |
| pH 8.0 100× | | | |
| dH₂O | — | — | 247.5 ml |

Filter (45 μm) Sterilize and store at Room Temperature

4.5x SSC/0.15% Sarkosyl Hybridization Buffer
(MICROSPHERE DILUENT)

| | | Final | Amount/ |
|---|---|---|---|
| Reagent | Catalog Number | Concentration | 50 ml |
| 20x SSC | Cambrex US51232 | 4.5x | 11.25 ml |
| (3M Sodium | | | |
| chloride, 0.3M | | | |
| Sodium citrate | | | |
| dehydrate, pH 7.0) | | | |
| 20% Sarkosyl | — | 0.15% | 0.375 ml |
| dH₂O | — | — | 38.375 ml |

Filter (45 μm) Sterilize and store at Room Temperature

3x SSC/0.1% Sarkosyl/1 mg/ml Casein Stringent Wash Buffer

| | | Final | Amount/ | |
|---|---|---|---|---|
| Reagent | Catalog Number | Concentration | 50 ml | |
| 20x SSC | Cambrex US51232 | 3x | 7.5 | ml |
| 20% Sarkosyl | — | 0.1% | 0.250 | ml |
| 50 mg/ml Casein | VWR | — | 1 | ml |
| (pH7.2) | BDHA440203H | | | |
| dH₂O | — | — | 41.25 | ml |

Filter (45 μm) Sterilize and store at 4° C.

1x SSC/0.1% Sarkosyl/1 mg/ml Casein Wash Buffer

| | | Final | Amount/ | |
|---|---|---|---|---|
| Reagent | Catalog Number | Concentration | 50 ml | |
| 20x SSC | Cambrex US51232 | 1x | 2.5 | ml |
| 20% Sarkosyl | — | 0.1% | 0.250 | ml |
| 50 mg/ml Casein | VWR | — | 1 | ml |
| (pH7.2) | BDHA440203H | | | |
| dH₂O | — | — | 46.25 | ml |

Filter (45 μm) Sterilize and store at 4° C.

### B. Beads

1. Bead types used are L100-C123-01 up to L100-C172-01 (Luminex™ Corp., Austin, Tex.).

### C. Probes (See Examples)

1. Probes were supplied by Eurogentec (Seraing, Belgium)

### D. Equipment

| Equipment | Type |
|---|---|
| Thermocycler | ABI GeneAmp PCR system 9700 |
| Thermo mixer | Eppendorf Thermomixer comfort |
| Water bath | GFL 1001 |
| Incubation Oven | Memmert U25U |
| Luminex ™ | Luminex ™ X100 |

### Methods & Protocols:

### I. Probe Coupling

1. Bring a fresh aliquot of −20° C., desiccated Pierce EDC [1-Ethyl-3-[dimethylaminopropyl]carbodiimid hydrochlorid] powder to room temperature.
2. Resuspend the amine-substituted oligonucleotide (“probe” or “capture” oligo) to 0.2 mM (0.2 nmol/μl) in dH₂O.
3. Resuspend the stock microspheres by vortex and sonication for approximately 20 seconds.
4. Transfer 5.0×10⁶ of the stock microspheres to a USA Scientific microfuge tube.
5. Pellet the stock microspheres by microcentrifugation at ≧8000×g for 1-2 minutes.
6. Remove the supernatant and resuspend the pelleted microspheres in 50 μl of 0.1 M MES, pH 4.5 by vortex and sonication for approximately 20 seconds.
7. Prepare a 1:10 dilution of the 0.2 mM capture oligo in dH₂O (0.02 nmol/μl).
8. Add 2 (0.04 nmol) of the 1:10 diluted capture oligo to the resuspended microspheres and mix by vortex.
9. Prepare a fresh solution of 20 mg/ml EDC in dH2O. Dissolve 10 mg EDC in 500 μl dH2O, maximally 1 minute before use. Aliquots of 10 mg EDC (powder) were stored dry at −80° C. packed together with silica gel.
10. One by one for each reaction, add 2.5 μl of freshly prepared 20 mg/ml EDC to the microspheres and mix by vortex (Note: The aliquot of EDC powder should now be discarded).
11. Incubate for 30 minutes at room temperature in the dark.
12. Prepare a second fresh solution of 20 mg/ml EDC in dH2O.
13. One by one for each reaction, add 2.5 μl of fresh 20 mg/ml EDC to the microspheres and mix by vortex (Note: The aliquot of EDC powder should now be discarded).
14. Incubate for 30 minutes at room temperature in the dark.
15. Add 1.0 ml of 0.02% Tween-20 to the coupled microspheres.
16. Pellet the coupled microspheres by microcentrifugation at ≧8000×g for 1-2 minutes.
17. Remove the supernatant and resuspend the coupled microspheres in 1.0 ml of 0.1% SDS by vortex.
18. Pellet the coupled microspheres by microcentrifugation at ≧8000×g for 1-2 minutes.
19. Remove the supernatant and resuspend the coupled microspheres in 100 μl of TE, pH 8.0 by vortex and sonication for approximately 20 seconds.
20. Pellet the coupled microspheres by microcentrifugation at ≧8000×g for 1-2 minutes.
21. Remove the supernatant and resuspend the coupled microspheres in 100 μl of TE, pH 8.0 by vortex and sonication for approximately 20 seconds.
22. Enumerate the coupled microspheres by hemacytometer:
   a. Dilute the resuspended, coupled microspheres 1:100 in dH₂O.
   b. Mix thoroughly by vortex.
   c. Transfer 10 μl to the hemacytometer.
   d. Count the microspheres within the 4 large squares of the hemacytometer grid.
   e. Microspheres/μl=(Sum of microspheres in 4 large squares)×2.5×100 (dilution factor). (Note: maximum is 50,000 microspheres/μl)
23. Store coupled microspheres refrigerated at 2-10° C. in the dark.

### II. Optimized Hybridization & Wash Protocol

1. Select the appropriate oligonucleotide-coupled microsphere sets.
2. Resuspend the microspheres by vortex and sonication for approximately 20 seconds.
3. Prepare a Working Microsphere Mixture by diluting coupled microsphere stocks to 150 microspheres of each set/μl in 4.5×SSC/0.15% Sarkocyl Hybridization Buffer (Note: 33 μl of Working Microsphere Mixture is required for each reaction).
4. Mix the Working Microsphere Mixture by vortex and sonication for approximately 20 seconds.
5. To each sample or background well, add 33 μl of Working Microsphere Mixture.
6. To each background well, add 17 μl TE, pH 8.
7. To each sample well add amplified biotinylated DNA and TE, pH 8.0 to a total volume of 17 μl (Note: 4 μl of a robust 50 μl PCR reaction is usually sufficient for detection).
8. Mix reaction wells gently by pipetting up and down several times.
9. Incubate at 95-100° C. for 5 minutes to denature the amplified biotinylated DNA in a thermocycler.
10. Incubate the reaction plate at 60° C. for 3 minutes in a thermocylcer.
11. Transfer the reaction plate to a thermomixer pre-heated at hybridization temperature (Note: An 8-channel pipettor can be used to transfer the reactions in 8 wells simultaneously).
12. Incubate the reaction plate at hybridization temperature for 15 minutes and 500 rpm
13. During incubation, prepare the Millipore filter plate by rinsing with distilled water. Next, fill each well of the filter plate with 200 μl 3×SSC/0.1% Sarkosyl/1 mg/ml Casein wash Buffer at hybridization temperature and place it in an oven at the hybridization temperature.
14. During incubation, prepare fresh reporter mix by diluting streptavidin-R-phycoerythrin to 2 μg/ml in 3×SSC/0.1% Sarkocyl/1 mg/ml Casein stringent wash buffer (Note: 75 μl of reporter mix is required for each reaction), and place it in an oven or water bath at the hybridization temperature.
15. Terminate the hybridization reaction by transferring the entire reaction to the filter plate containing wash buffer at hybridization temperature
16. After transfer, wash the filter plate twice with 100 μl 3×SSC/0.1% Sarkocyl/1 mg/ml Casein stringent wash buffer at hybridization temperature by intervening vacuum filtration
17. Add 75 μl of reporter mix to each well and mix gently by pipetting up and down several times.
18. Incubate the reaction plate at hybridization temperature for 15 minutes
19. Terminate the incubation by vacuum filtration.
20. Wash twice with 100 μl 1×SSC/0.1% Sarkosyl/1 mg/ml Casein wash buffer at room temperature by intervening vacuum filtration
21. Dissolve a reaction in 100 μl 1×SSC/0.1% Sarkosyl/1 mg/ml Casein wash buffer at room temperature
22. Analyze 50 μl at room temperature on the Luminex™ 100 analyzer according to the system manual.

### III. Read-Out

1. Data was read out using the Luminex™ 100 IS version 2.3 software
2. During measurement the following parameters are used:
   a. Sample volume: 50 μl
   b. Sample timeout: 60 sec.
   c. XY heater temp (° C.): 35
   d. Doublet Discriminator Gate:
      i. Low Limit: 8000
      ii. High Limit: 18500
   e. Statistic: median
      IV. Data management
1. Data was saved in a raw CSV file (comma delimited *.csv) containing all standard output as provided by the Luminex™100 IS2.3 software.
2. The median signals obtained were transferred to an Excel file for calculation of the target to probe ratio and signal to noise ratio (see also layout and calculations).

The present invention addresses different items of the Luminex™ procedure, including the optimization of the probe design and optimization of the test protocol.

In the following text, data will be presented in the order of the work-flow, as outlined in FIG. 2.

FIG. 2. General schematic overview of the adapted work-flow

### Presentation of Results in the Examples (Layout and Calculations)

The examples and claims involved are specified and explained as follows. Results are mainly presented as tables containing raw data (MFI=median fluorescent intensity), variables (e.g. temperature), probes, and targets as analyzed, calculations, and remarks. The calculations include a target to probe ratio (% target/probe) and a signal to noise ratio (signal/noise).

The target to probe ratio is calculated per probe and displays each of the signals as a percentage of the positive control which is set at 100% (see also example Table 12).

The signal to noise ratio is also calculated per probe. Each signal is divided by the median of all signals obtained (see also example Table 13).

Both the target to probe ratio and signal to noise ratio give a good overall indication on signal intensity and specificity.

Certain examples use probes from the SPF10 primer and probe sets, described in EP1012348, herein incorporated fully by reference. This patent provides a technical background to the techniques used in the present patent application.

The SPF10 primer set generates small amplimers of only 65 by in length, with an interprimer region of 22 nucleotides. This severely limits the possibilities to position the probes with respect to the different mismatches between all HPV genotypes.

### Example 1

### Objective

To examine if maintenance of the hybridization temperature after the hybridization step has a significant positive effect on signal specificity.

### Introduction:

After hybridization between the immobilized probe on the bead and the denatured target sequence in solution, the unbound material needs to be washed away before incubation with the reporter reagent Streptavidin-R-phycoerythrin (PE). This is achieved by using a filter plate (MSBVN12, Millipore), where the beads and all attached molecules are separated from molecules free in solution. The reaction volume is small and therefore vulnerable to rapid temperature changes in its environment. We examined the effect of changes in temperature after hybridization temperature.

### Materials and Methods:

The effect of incubation at a temperature lower than the hybridization on the Luminex™ signal was investigated using the SPF₁₀ model system.

A Luminex™ bead was used, carrying a probe for HPV 31 (probe 31SLPr31, see table 1a). This probe is specific for identification of HPV 31 sequences amplified with the SPF₁₀ primer set. To assess any cross-reactivity amplimers of HPV44 and HPV16 were used. Target sequences of HPV 31 and HPV 44 differ in 1 position and target sequences of sequences of HPV 31 and HPV 16 differ in 4 positions (Table 1b).

Hybridization was performed at 50° C. and assays were run in duplicate. Subsequently, one set of reactions were treated according to the standard protocol and the beads were immediately washed in the filter plate at 4° C. The duplicate set of reactions was first incubated at room temperature (RT) for 1 minute before starting the same standard wash at 4° C. In contrast to Wallace et al (2005), wash buffer was added after the samples were transferred to the filter plate (see also example 2).

### Results:

Results are shown in the Table 1c. As demonstrated, incubation at RT for just 1 minute after hybridization and before the stringent wash causes an increase in signal but also decreases specificity (shown by higher signals observed for HPV44). This can be explained by the reduction in stringency, caused by the brief temperature drop after hybridization.

### Conclusion:

The temperature of the reaction should be maintained after the hybridization step. After hybridization the beads should be washed as quickly as possible without any delay to prevent any decrease in temperature.

### Example 2

### Objective

To examine if a dilution wash, immediately after hybridization, has a significant positive effect on the specificity of the signal.

### Introduction:

The standard Luminex™ assay procedure comprises a risk for introducing aspecific binding if the washing is not immediately following the hybridization step (see also example 1). To minimize this risk the dilution of the sample immediately after hybridization was examined.

### Materials and Methods:

To investigate this effect, a mixture of two Luminex™ beads was used, one bead carrying a probe for HPV 31 (name: 31SLPr31, see table 2a) and another bead carrying HPV 51 (name: 51SLPr2, see table 2a). These probes are specific for identification of HPV 31 and HPV 51 sequences amplified with the SPF₁₀ primer set, respectively. To observe possible cross reactivity with 31SLPr31 amplimers of HPV44 and HPV16 were used. Target sequences of HPV 31, and HPV 44 and 16 differ in 1 and 4 positions, respectively (Table 2b). To observe possible cross reactivity with 51SLPr2 amplimers of HPV33 and HPV16 were used. Target sequences of HPV 51 and HPV 44 and 16 each differ in 4 positions (Table 2c).

Hybridization was performed at 50° C., using the standard protocol.

Subsequently, the first set of reactions was immediately washed in the filter plate at 4° C. without any additional wash. In contrast to Wallace et al (2005), wash buffer was added after the samples were transferred to the filter plate.

The effect of an additional direct and indirect dilution wash procedure, immediately following the hybridization step was investigated as follows. For the direct and indirect procedures a wash buffer (3×SSC/0.1% Sarkosyl/1 mg/ml Casein. This is the stringent Wash Buffer) was used at 50° C.

The second set of beads was washed by the direct procedure. The direct procedure comprises a dilution of the hybridization mix (50 μl) with 200 μl of wash buffer at hybridization temperature in the thermocycler followed by a transfer of the entire diluted sample to the filter plate.

The third hybridization reaction was washed by the indirect procedure. The indirect procedure comprises a dilution by a rapid transfer of the 50 μl of the hybridization mix to the filter plate which was already prefilled with 200 μl of wash buffer at hybridization temperature (see also Wallace et al, 2005).

### Results:

Results are shown in the table 2d. Both additional wash procedures yield a decrease of the absolute signal, as compared to the standard procedure, but at the same time the specificity of the signal increases significantly. There were no significant differences between the direct and indirect wash procedures. In practice, the direct dilution wash in the thermocycler is less practical, and therefore, the indirect dilution wash procedure is preferred.

### Conclusion:

The use of an additional dilution-wash step after hybridization has a significant positive effect on signal specificity. For practical reasons, the indirect dilution wash procedure is preferred.

### Example 3

### Objective

To examine if maintenance of the hybridization temperature during the stringent wash before incubation with Streptavidin-R-phycoerythrin, has a significant positive effect on the signal specificity.

### Introduction:

The negative effect of a temperature drop after stringent hybridization, as described above, implies that temperature of the stringent wash itself also can be of influence. Therefore, the effect of the stringent wash temperatures at 50° C., RT or 4° C. was investigated.

### Materials and Methods:

The effect of different stringent wash buffer temperatures, following the hybridization step before incubation with Streptavidin-R-phycoerythrin was investigated using the SPF₁₀ model system as follows.

To investigate this effect, a Luminex™ bead was used, carrying a probe for HPV 31 (name: 31SLPr31, see table 3a). This probe is specific for identification of HPV 31 sequences amplified with the SPF₁₀ primer set. To observe possible cross reactivity with 31SLPr31 amplimers of HPV44 and HPV16 were used. Target sequences of HPV 31 and HPV 44 and 16 differ in 1 and 4 positions, respectively (Table 3b).

Hybridization was performed at 50° C. Subsequently, the set of reactions were transferred to a filter plate containing wash buffer at 50° C., RT, or 4° C., respectively.

### Results:

Results are shown in table 3c. The absolute level of the positive control signal does not differ between 50° C. and RT, and is slightly decreased after washing at 4° C. However, washing at 50° C. results in a significant increase of signal specificity, whereas washing at RT or 4° C. results in a decrease of signal specificity. Therefore, an indirect dilution wash procedure at hybridization temperature of 50° C. is preferred.

### Conclusion:

Maintenance of the hybridization temperature during the stringent wash before incubation with Streptavidin-R-phycoerythrin, has a significant effect on the signal specificity.

### Example 4

### Objective

To examine if the use of a thermomixer has a significant positive effect on signal intensity.

### Introduction:

The kinetics of a hybridization reaction can be influenced by mixing the components during the reaction.

Therefore we investigated the influence of using a thermomixer during hybridization.

### Materials and Methods:

The effect of diffusion kinetic using a thermomixer during hybridization was investigated using the MPF model system as follows.

Two Luminex™ beads were used, carrying either a probe for HPV18 (name: 18MLPr7, see table 4a) or HPV51 (name: 51MLPr2, see table 4a). These probes are specific for identification of HPV18 and HPV51 sequences amplified with the MPF primer set.

The two beads were mixed and hybridized with MPF amplimers of HPV18 and HPV 51. Target sequences of HPV18 and HPV51 differ in 7 positions (Table 4b and c). Reactions were tested in duplicate.

One reaction was denatured and hybridized in a thermocycler, without shaking. (see also Wallace et al, 2005)

The duplicate reaction was denatured in a thermocycler for denaturation, and immediately transferred to a thermomixer for hybridization. Hybridization was performed at 50° C. Subsequently, the beads were immediately washed in the filter plate at 50° C., using the optimized hybridization and wash protocol.

Results: Results are shown in table 4d. Use of a thermo-mixer significantly increases the absolute signal of the positive control, whereas the background remained unaffected. This resulted in an overall increase of signal specificity.

These results demonstrate that the signal intensity will be increased (improved) by using a thermo-mixer.

### Conclusion:

The use of a thermo-mixer has a significant positive effect on the signal intensity and specificity.

### Example 5

### Objective

To examine if incubation with Streptavidin-R-phycoerythrin at the hybridization temperature has a significant positive effect on the signal intensity.

### Introduction:

In general, temperature affects the kinetics of any reaction, including the detection of hybrids with the reporter PE. Therefore, the influence of temperature for PE incubation and the subsequent wash was investigated.

### Materials and Methods:

Luminex™ beads were used, carrying a probe for HPV51 (name: 51SLPr2, see table 5a). This probe is specific for identification HPV51 sequences amplified with the SPF₁₀ primer set. To observe possible cross reactivity with this probe, SPF10 amplimers of HPV33 and HPV16 were used. Target sequences of HPV 51, HPV33 and HPV16 differ at 4 positions (Table 5b).

Hybridization was performed at 50° C. in two replicates, using the optimized hybridization and wash protocol outlined herein. After stringent wash, one set of reactions was incubated with PE at 50° C. (see also Wallace et al, 2005), and the other set was incubated with PE at RT. Subsequently, the beads were washed in a filter plate at 50° C.

In another experiment, hybridization was performed at 50° C. in two replicates, using the optimized hybridization and wash protocol. After stringent wash, all reactions were incubated with PE at 50° C. (see also Wallace et al, 2005). After PE incubation at 50° C., one set of reactions was washed at 50° C. (see also Wallace et al, 2005), and the duplicate set was washed at RT.

### Results:

PE incubation at different temperatures had a significant effect, as shown in table 5c. PE incubation at the hybrizidation temperature of 50° C. results in higher absolute signals, as compared to PE incubation at RT. However, the specificity of the signal did not differ significantly.

Therefore, incubation at with Streptavidin-R-phycoerythrin at hybrizidation temperature is preferred. In contrast, washing at RT or hybridization temperature after incubation did not have a significant effect, although this may be more practical in some situations.

The influence of temperature on the washing step after PE incubation is not significant. Both the absolute signal as well as the specificity appear not to be affected by the temperature of the wash.

### Conclusion:

Maintenance of the hybridization temperature during incubation with Streptavidin-R-phycoerythrin, has a significant effect on the signal intensity but not on the signal specificity.

The temperature of the wash after PE incubation has no significant effect.

### Example 6

### Objective

To examine whether clogging of Luminex™ sampling probe can be prevented by a final wash with 1×SSC.

### Introduction:

In our optimized hybridization and wash protocol hybridization is performed in 3×SSC. At this concentration SSC does clog the Luminex™ sampling probe seriously obstructing processing of the samples. Therefore, the influence of a lower SSC concentration was investigated for a final wash.

### Results:

Initially we tried to maintain the SSC concentration of the hybridization. However, as a final wash with 3×SSC introduced a serious clogging of the Luminex™ sampling probe, no significant data could be produced. Simply performing this wash step with 1×SSC did result in significant data. Therefore, due to lacking data, a comparison by data can not be shown. Other SSC concentrations have not been investigated.

### Conclusion:

A final wash with 1×SSC prevents clogging of the Luminex™ sampling probe.

### Example 7

### Objective

To examine if storage after the final wash at 4° C. for at least 4 days of samples that are ready for measuring has any significant effect on the signal.

### Introduction:

To increase flexibility on the work floor we analyzed several steps with respect to the direct hybridization test protocol using the Luminex™ system. One procedure tested in particular is storage in between two steps of the direct hybridization procedure. Therefore, we investigated the influence of storage at 4° C.

### Materials and Methods:

The effect of storage at 4° C. after the final washing procedure was investigated using the SPF10 model system as follows.

To investigate this effect, Luminex™ beads were used, carrying a probe for HPV51 (name: 51SLPr2, see table 7a). This probe is specific for identification HPV51 sequences 0.0 amplified with the SPF₁₀ primer set. To observe possible cross reactivity with 51SLPr2 amplimers of HPV31 were used. Target sequences of HPV 51 and, HPV31 differ in 4 positions (Table 7b).

Following the final wash procedure, sets of reactions were stored at 4° C., for 0, 4, 24, and 96 hrs, respectively. Next, these reaction sets were measured at RT.

### Results:

Results are shown in table 7c. As demonstrated, storage after the final wash step does not affect signal intensity or specificity. Nevertheless, storage as such seems to introduce a very slight improve in raw signal intensity over time. Therefore, storage after the final wash step can be introduced if necessary for a maximum of 4 days, maintaining the original signal.

### Conclusion:

Storage after the final wash step has no significant effect on signal intensity and signal specificity, increasing flexibility on the work floor.

### Probe (Spacer) Design—Introduction

The key principle of the Luminex™ system is the immobilization of specific oligonucleotide probe on the surface of a microbead, which serves as a unique label, due to the color composition of the individual bead types.

At the molecular scale, the bead is much bigger that the specific oligonucleotide probe. Consequently, the specific probe sequence is positioned very closely to the surface of the Luminex™ bead. This probe location may not be the optimal for hybridization kinetics between the immobilized probe and the target molecules in solution, due to steric hindrance and various bead surface effects, such as surface hydrophobicity.

The following examples describe a number of approaches to change the positioning of the probe onto the bead surface, in order to optimize the hybridization kinetics between probe and target.

The following variants in probe design were tested:
1. Use of a carbon spacer of variable length
2. Use of an additional oligonucleotide spacer of variable length
3. Use of an oligonucleotide spacer of variable composition

The probe has three distinct regions, with different functions;
1. the coupling group, such as an NH2 group, which permits covalent coupling of the probe to the bead surface;
2. the spacer, which may serve (a) to create a distance between the bead surface and the specific probe sequence and/or (b) to position the specific probe more in a hydrophilic environment; and
3. the actual target-specific probe sequence. For this part of the probe, the normal parameters in the art, such as probe composition and length apply.

### Example 8

### Objective

To determine the effect of the use of a carbon spacer of variable length.

### Materials and Methods:

Luminex™ beads were used, carrying either a probe for HPV51 with a C₁₂ spacer (name: 51SLPr2, see table 8a) or a C₁₈ spacer (name: 51SLPr2C₁₈, see table 8a). These probes are specific for identification HPV51 sequences amplified with the SPF₁₀ primer set. To observe possible cross reactivity with these probes, amplimers of HPV33 were used. Target sequences of HPV 51 and HPV33 differ in 4 positions (Table 8b).

Results: Results are shown in table 8c. A C18 spacer resulted in a decrease in absolute signal, but the specificity was higher as compared to the C12 probe. This phenomenon was not only seen for 51SLPr2C₁₈, but also for other probes with a C₁₈ carbon spacer (e.g. 33SLPr21 C₁₈ Table 8a, c, and d).

### Conclusion:

The use of different carbon spacer lengths has a significant effect on signal specificity. With respect to for example 51SLPr2, the best probe contains a C₁₈ carbon spacer.

### Example 9

### Objective

To determine the effect of an oligonucleotide spacer of variable length.

### Materials and Methods:

Luminex™ beads were used, carrying a probe for HPV51 with a spacer of either 0, 10, 20, 30, or 40 Thymines (name: 51SLPr2, 51SLPr2T10, 51SLPr2T20, 51SLPr2T30, 51 SLPr2T40, see table 9a). Each bead type carried a distinct probe variant. These probes are specific for identification HPV51 sequences amplified with the SPF₁₀ primer set. To observe possible cross reactivity with these probes, amplimers of HPV33 were used. Target sequences of HPV51 and HPV33 differ in 4 positions (Table 9c).

Apart from the SPF10 model system this effect was also studied using the MPF model system as follows. Luminex™ beads were used, carrying a probe for HPV52 with a spacer of either 0, 20, 30, or 40 Thymines (name: 52MLPr2, 52MLPr2T20, 5MLPr2T30, 52MLPr2T40, see table 9b). Each bead type carried a distinct probe variant. These probes are specific for identification HPV52 sequences amplified with the MPF primer set. To observe possible cross reactivity with these probes, amplimers of HPV16 were used. Target sequences of HPV52 and HPV16 differ in 2 positions (Table 9d).

### Results:

Results are shown in table 9e and 9f. Elongation of the spacer with a thymine stretch significantly increases the absolute signal level. Also, the specificity is significantly increased, as compared to a spacer without an additional thymine spacer. Comparing the spacers with different lengths, a minimum of 20 thymine residues is required to yield an optimal signal (e.g. 51SLPr2). Overall, probes perform best when they contain a spacer of 40 nucleotides (e.g 51SLPr2, and 52MLPr2). Therefore this spacer length is preferred.

### Conclusion:

The use of different spacers has a significant effect not only on signal intensity, but also on specificity. With respect to 51SLPr2Tₙ, a good probe contains a spacer of at least 20 thymine nucleotides increasing both signal intensity and specificity. In general, a spacer length of at least 40 nucleotides performs best.

### Example 10

### Object

To determine whether use of a modified poly(T) spacer can prevent false-positive reactivity.

### Introduction:

It is well known that many Taq DNA polymerases add an additional A-nucleotide at the 3′ end of a synthesized strand. It is not known whether also multiple A's can be added to the 3′ end, thereby generating a subpopulation of molecules with an oligo-A tail at the 3′ end. Although such molecules will only represent a very small proportion of the total amount of PCR product, these molecules can result in false-negative result, due to the high sensitivity of the detection method. This is due to the fact that hybridization between such oligo-A stretches at the PCR-product and the poly(T) spacer of the probe.

This PCR artifact occurs in some samples, and is hard to reproduce at the PCR level. It appears to be dependent on very small fluctuations in reaction conditions. The background is very reproducible at the detection level, i.e. a PCR product generating background will do so very reproducibly.

This PCR artifact can also cause false-positive results on a line probe assay (LiPA) system, since this system also comprises T-tailed probes. In a LiPA assay this results in a weak equal (background) signal with all probes, irrespective of their specific sequence. Also in the Luminex™ system such weak background signal readouts have been observed. Therefore, the effect of a modified spacer was investigated.

### Materials and Methods:

Luminex™ beads were used, carrying either a probe for HPV18 with a T40 spacer, or a modified (TTG)13 spacer (name: 18MLPr7T40 and 18MLPr7(TTG)₁₃, see table 10a). These probes are specific for identification of HPV18 sequences amplified with the MPF primer set. The (TTG) triplet was chosen as an alternative spacer because it shows one of the worst theoretical binding efficiencies with poly (A).

To observe possible cross reactivity with 18MLPr7T40 and 18MLPr7(TTG)₁₃ amplimers derived from samples showing this false-positive background were used (designated nc8).

### Results:

Results are shown in table 10b.

A spacer of 13 “TTG” nucleotide triplets was clearly able to almost completely eliminate the background signal, which was observed for the T40 spacer.

### Conclusion:

The use of an alternative T-based spacer, such as (TTG)₁₃ has a significant positive effect on the signal specificity, eliminating false-positive signals induced by A-rich PCR artifacts.

### Example 11

### Object

To examine if positioning a Thymine based spacer at either the 5′- or 3′-end of a probe prohibits binding to an A-rich target region flanking the probe-target binding site.

### Introduction:

It is known that mismatches in the middle of a probe/target have the largest impact on its binding energy. Mismatches close to the sides of the binding region are more difficult to distinguish. In combination with the position of A-rich stretches flanking the probe/target binding region this may harm the selective strength of a probe. Therefore, we investigated the influence of the spacer position to minimize its binding to an A-rich target region flanking the probe-target binding site.

### Materials and Methods:

The effect of a spacer position at either the 5′- or 3′-end of a probe, positioned between the Luminex™ bead and the specific probe sequence was investigated using the MPF model system as follows.

To investigate this effect, Luminex™ beads were used, carrying a probe for HPV18 and HPV45 with a Thymine based spacer (name: 18MLPr7T40N5, 18MLPr7T40N3, 45MLPr8T40N5 and 45MLPr8T40N3, see table 11a). These probes are specific for identification of HPV18 and HPV45 sequences amplified with the MPF primer set, respectively. To observe possible cross reactivity with 18MLPr7T40ₙ amplimers of HPV39 were used. Target sequences of HPV18 and, HPV39 differ in 2 positions (Table 11b). To observe possible cross reactivity with 45MLPr8T40ₙ amplimers of HPV13, 39, and 40 were used. Target sequences of HPV45 and, HPV13, 39 and 40 differ in 3, 2, and 1 position, respectively (Table 11c).

### Results:

Results are shown in table 11d. As demonstrated, a spacer at the 3′-end of a probe instead of the 5′-end decreases its binding to an A-rich target region flanking the probe-target binding site, affecting the binding energy (dG) and melting temperature (Tms). The exclusion of these aspecific signals can be explained by binding of the target to the spacer and probe. These results suggest that the binding of a target to the spacer can hamper probe specificity, which should be prevented. In principle a likewise mechanism may be involved using a “TTG” nucleotide triplet spacer. Therefore, when using a Thymine based spacer, the stability of the probe:target hybrid can be increased by weak cross-hybridization between spacer and sequences adjacent to the specific target region, resulting in false-positive signal which should be taken into account for the probe design.

### Conclusion:

The position of a Thymine based spacer at either the 5′ or 3′ end of a probe can have a significant effect with respect to binding an A-rich target region flanking the probe-target binding site.

### Example 12

### HPV Probes Suitable for Use with Bead Based Approaches, Eg for Luminex Based Approaches

TABLE 14

| Name | Probe sequence |
|---|---|
| 16MLP4T40N3 | GAGCACAGGGCCAC(T)₄₀ |
| 18MLPr7T40N3 | TTACATAAGGCACAGG(T)₄₀ |
| 26MLP7T40N3 | GTTACAACGTGCACAG(T)₄₀ |
| 31MLPr6T40N3 | GGATGCAACGTGCTC(T)₄₀ |
| 33MLPr4T40N5 | (T)₄₀CATATTGGCTACAACGT |
| 35MLPr6T40N3 | GTGCACAAGGCCATA(T)₄₀ |
| 39MLPr4T40N5 | (T)₄₀GCCTTATTGGCTACATAA |
| 45MLPr6T40N5 | (T)₄₀ggtGTTACATAAGGCCCAG |
| 45MLPr8T40N3 | CCAGGGCCATAACAAg(T)₄₀ |
| 51MLPr2T40N5 | (T)₄₀TTATTGGCTCCACCGT |
| 52MLPr2T40N5 | (T)₄₀CCGTACTGGTTACAACGa |
| 53MLPr6T40N5 | (T)₄₀ATATTGGCTGCAACGT |
| 56MLPr4T40N5 | (T)₄₀GGCCCAAGGCCATAATAA |
| 58MLPr1T40N5 | (T)₄₀CTTATTGGCTACAGCGT |
| 58MLPr5T40N3 | ACAGCGTGCACAAGG(T)₄₀ |
| 59MLPr3T40N5 | (T)₄₀CAAGGCTCAGGGTTTAA |
| 66MLPr6T40N3 | TGCACAGGGCCATA(T)₄₀ |
| 66MLPr7T40N3 | TGCAACGTGCACAG(T)₄₀ |
| 68MLPr8T40N5 | (T)₄₀CTGCACAAGGCACAG |
| 68MLPr10T40N3 | GCACAAGGCACAGG(T)₄₀ |
| 70MLPr4T40N5 | (T)₄₀CCTATTGGTTGCATAAGG |
| 82MLPr3T40N3 | ATTGGTTGCATCGCG(T)₄₀ |

In one aspect of the invention any 1, 2, 3, 4, 5, 6, 7, 8, 9, 10, 11, 12, 13, 14, 15, 16, 17, 18, 19, 20, 21 or all 22 all the above probes may be used in a bead-based multiplex reaction under identical conditions for simultaneous detection of any HPV target DNA present in a sample. Such bead sets are suitable for use in the optimized reaction scheme outlined above. An additional polycarbon spacer may be incorporated. As such the invention relates to any probe set comprising, 2, 3, 4, 5, 6, 7, 8, 9, 10, 11, 12, 13, 14, 15, 16, 17, 18, 19, 20, 21 or all 22 all the above probes.

### General Conclusion

We hypothesized that steric hindrance and hydrophobic repulsion results in sub-optimal mismatch discrimination at the extremity of a hybrid. In addition, this steric hindrance was thought to reduce sensitivity because a target is bound less optimal to a probe. Therefore, inclusion of a spacer in the probe design was shown to prevent steric hindrance and therefore eliminates cross-hybridization and increases the sensitivity. We observed that this indeed results in a higher specificity and sensitivity.

Although this spacer increased the probe performance significantly, some cross reactions still were observed. Most of these cross reactions were found to have a single mismatch near the end of the probe (target binding region). Sometimes such a cross reaction was seen in combination with an A-rich region flanking the probe binding region of a target. We therefore hypothesized that such an A-rich region may bind to a portion of the T-stretch of the spacer and thereby increase cross reactivity. Initially, we then designed the probe with a spacer at the other end (3′ instead of 5′). However, redesign of the spacer, taking the sequences flanking the probe binding region into account, could be an alternative.

We have observed that in a PCR sometimes an artificial product is generated, which tends to bind to all probes. This product may contain a number of A-residues at the 3′ end (this is a known activity of several Taq polymerases) therefore has an increased affinity for the T-based spacer. A-T hybridization could result in increased cross-reactivity, leading to false positive hybridization results.

To gain more insight in this phenomenon we designed several probes with a spacer comprising TTG-triplets (e.g. (TTG)₁₃). We calculated that in particular this triplet would be most repulsive and diminish binding of the artificial PCR product with A-rich sections flanking the probe target region. We observed that the overall aspecific binding of this artificial PCR was decreased by the TTG-based spacer. A TTG-based spacer may also diminish the binding of the probe flanking region, and increase its specificity.
In summary:
   Steric hindrance and hydrophobic repulsion results in sub optimal mismatch discrimination at the extremity of a hybrid
   Inclusion of a spacer in the probe design prevents steric hindrance and therefore eliminates cross-hybridization, which results in a higher specificity and sensitivity.
   An A-rich region flanking the probe binding region of a target can bind to a T-stretch of the spacer and increase cross reaction.
   Sometimes, in a PCR an artificial product is generated, containing A-stretches at the 3′ end, which tends to bind to all probes.
   This product is A-rich and therefore has an increased affinity for the T-based spacer.
   This phenomenon can be decreased by a TTG-based spacer, diminishing the a-specific binding of the probe flanking region, and increase its specificity.

### LITERATURE REFERENCES

Cowan L S, Diem L, Brake M C, Crawford J T. Related Articles. Transfer of a Mycobacterium tuberculosis genotyping method, Spoligotyping, from a reverse line-blot hybridization, membrane-based assay to the Luminex multianalyte profiling system. J Clin Microbiol. 2004 January; 42(1):474-7.
Dunbar S A. Applications of Luminex™ xMAPtrade mark technology for rapid, high-throughput multiplexed nucleic acid detection. Clin Chim Acta. 2005 Aug. 12; [Epub ahead of print]
Taylor J D, Briley D, Nguyen Q, Long K, Iannone M A, Li M S, Ye F, Afshari A, Lai E, Wagner M, Chen J, Weiner M P. Flow cytometric platform for high-throughput single nucleotide polymorphism analysis. Biotechniques. 2001 March; 30(3):661-6, 668-9.
de Villiers E M, Fauquet C, Broker T R, Bernard H U, zur Hausen H. Classification of papillomaviruses. Virology. 2004 Jun. 20; 324(1):17-27. Review.
Wallace J, Woda B A, Pihan G. Facile, comprehensive, high-throughput genotyping of human genital papillomaviruses using spectrally addressable liquid bead microarrays. J Mol Diagn. 2005 February; 7(1):72-80.

### Test Example 1

TABLE 1a
31SLPr31 = SPF₁₀ probe 31 version 31,
C₁₂ = a stretch of 12 carbon atoms

| Name | Probe composition |
|---|---|
| 31SLPr31 | NH₂—C₁₂-GGCAATCAGTTATTTG |

TABLE 1b
Identical nucleotides are indicated by a “-”.

| | Alignment | |
|---|---|---|
| Target | with probe 31SLPr31 | Number of mismatches |
| HPV 31 | GGCAATCAGTTATTTG | 0 |
| HPV 44 | --A------------- | 1 |
| HPV 16 | --T-C-AC-------- | 4 |

TABLE 1c

| | Hybridized to | Temperature after | | target/ | Signal/ | | |
|---|---|---|---|---|---|---|---|
| Probe | target | hybridization (° C.) | Signal (MFI) | probe (%) | noise | Remark | Exp |
| 31SLPr31 | SPF₁₀ HPV31 | 50 | 4457 | 100 | 48 | Specific | ID28 |
| 31SLPr31 | SPF₁₀ HPV44 | 50 | 1279 | 29 | 14 | Cross reaction | ID28 |
| 31SLPr31 | SPF₁₀ HPV16 | 50 | 19 | <1 | <1 | Negative | ID28 |
| 31SLPr31 | SPF₁₀ HPV31 | RT | 7544 | 100 | 13 | Specific | ID27 |
| 31SLPr31 | SPF₁₀ HPV44 | RT | 3783 | 50 | 6 | Cross reaction | ID27 |
| 31SLPr31 | SPF₁₀ HPV16 | RT | 24 | 1 | <1 | Negative | ID27 |

### Tables Example 2

TABLE 2a
31SLPr31 = SPF₁₀ probe 31 version 31,
C₁₂ = a stretch of 12 carbon atoms

| Name | Probe composition |
|---|---|
| 31SLPr31 | NH₂—C₁₂-GGCAATCAGTTATTTG |
| 51SLPr2 | NH₂—C₁₂-CTATTTGCTGGAACAATC |

TABLE 2b
Identical nucleotides are indicated by a “-”.

| | | Number |
|---|---|---|
| Target | Alignment with probe 31SLPr31 | of mismatches |
| HPV 31 | GGCAATCAGTTATTTG | 0 |
| HPV 44 | --A------------- | 1 |
| HPV 16 | --T-C-AC-------- | 4 |

TABLE 2c
Identical nucleotides are indicated by a “-”.

| | | Number |
|---|---|---|
| Target | Alignment with probe 51SLPr2 | of mismatches |
| HPV 51 | CTATTTGCTGGAACAATC | 0 |
| HPV 33 | T------T---GG----- | 4 |
| HPV 16 | -------T---GGT--C- | 4 |

TABLE 2d

| | | Add. wash | Signal | target/ | Signal/ | | |
|---|---|---|---|---|---|---|---|
| Probe | Hybridized to target | procedure | (MFI) | probe (%) | noise | Remark | Exp |
| 31SLPr31 | SPF₁₀ HPV31 | None | 4457 | 100 | 48 | Specific | ID28 |
| 31SLPr31 | SPF₁₀ HPV44 | None | 1279 | 29 | 14 | Cross reaction | ID28 |
| 31SLPr31 | SPF₁₀ HPV16 | None | 19 | <1 | <1 | Negative | ID28 |
| 31SLPr31 | SPF₁₀ HPV31 | Direct | 2765 | 100 | 41 | Specific | ID31 |
| 31SLPr31 | SPF₁₀ HPV44 | Direct | 117 | 4 | 2 | Negative | ID31 |
| 31SLPr31 | SPF₁₀ HPV16 | Direct | 20 | 1 | <1 | Negative | ID31 |
| 31SLPr31 | SPF₁₀ HPV31 | Indirect | 3843 | 100 | 171 | Specific | ID32 |
| 31SLPr31 | SPF₁₀ HPV44 | Indirect | 25 | 1 | 1 | Negative | ID32 |
| 31SLPr31 | SPF₁₀ HPV16 | Indirect | 15 | <1 | 1 | Negative | ID32 |
| 51SLPr2 | SPF₁₀ HPV51 | None | 2316 | 100 | 201 | Specific | ID28 |
| 51SLPr2 | SPF₁₀ HPV33 | None | 631 | 27 | 55 | Cross reaction | ID28 |
| 51SLPr2 | SPF₁₀ HPV16 | None | 11 | <1 | 1 | Negative | ID28 |
| 51SLPr2 | SPF₁₀ HPV51 | Direct | 2057 | 100 | 110 | Specific | ID31 |
| 51SLPr2 | SPF₁₀ HPV33 | Direct | 432 | 21 | 23 | Cross reaction | ID31 |
| 51SLPr2 | SPF₁₀ HPV16 | Direct | 18 | 1 | 1 | Negative | ID31 |
| 51SLPr2 | SPF₁₀ HPV51 | Indirect | 1571 | 100 | 209 | Specific | ID32 |
| 51SLPr2 | SPF₁₀ HPV33 | Indirect | 354 | 23 | 47 | Cross reaction | ID32 |
| 51SLPr2 | SPF₁₀ HPV16 | Indirect | 7 | <1 | 1 | Negative | ID32 |

### Tables Example 3

TABLE 3a
31SLPr31 = SPF₁₀ probe 31 version 31,
C₁₂ = a stretch of 12 carbon atoms

| Name | Probe composition |
|---|---|
| 31SLPr31 | NH₂—C₁₂-GGCAATCAGTTATTTG |

TABLE 3b
Identical nucleotides are indicated by a “-”.

| | | Number |
|---|---|---|
| Target | Alignment with probe 31SLPr31 | of mismatches |
| HPV 31 | GGCAATCAGTTATTTG | 0 |
| HPV 44 | --A------------- | 1 |
| HPV 16 | --T-C-AC-------- | 4 |

TABLE 3c

| | | Wash temp | Signal | target/ | Signal/ | | |
|---|---|---|---|---|---|---|---|
| Probe | Hybridized to target | (° C.) | (MFI) | probe (%) | noise | Remark | Exp |
| 31SLPr31 | SPF₁₀ HPV31 | 50 | 5747 | 100 | 162 | Specific | ID90 |
| 31SLPr31 | SPF₁₀ HPV44 | 50 | 56 | 1 | 2 | Negative | ID90 |
| 31SLPr31 | SPF₁₀ HPV16 | 50 | 20 | <1 | <1 | Negative | ID90 |
| 31SLPr31 | SPF₁₀ HPV31 | RT | 5701 | 100 | 33 | Specific | ID86 |
| 31SLPr31 | SPF₁₀ HPV44 | RT | 2422 | 42 | 14 | Cross react | ID86 |
| 31SLPr31 | SPF₁₀ HPV16 | RT | 13 | <1 | <1 | Negative | ID86 |
| 31SLPr31 | SPF₁₀ HPV31 | 4 | 4889 | 100 | 44 | Specific | ID34 |
| 31SLPr31 | SPF₁₀ HPV44 | 4 | 417 | 9 | 4 | Cross react | ID34 |
| 31SLPr31 | SPF₁₀ HPV16 | 4 | 33 | 1 | <1 | Negative | ID34 |

### Test Example 4

TABLE 4a
18MLPr7 = MPF probe 18 version 7,
C₁₂ = a stretch of 12 carbon atoms

| Name | Probe composition |
|---|---|
| 18MLPr7T40 | NH₂—C₁₂-(T)₄₀-TTACATAAGGCACAGG |
| 51MLPr2T40 | NH₂—C₁₂-(T)₄₀-TTATTGGCTCCACCGT |

TABLE 4b
Identical nucleotides are indicated by a “-”.

| | Alignment with probe | Number of |
|---|---|---|
| Target | 18MLPr7 | mismatches |
| HPV18 | TTACATAAGGCACAGG | 0 |
| HPV51 | C-C--CCGT--G---- | 7 |

TABLE 4c
Identical nucleotides are indicated by a “-”.

| | Alignment with probe | Number of |
|---|---|---|
| Target | 51MLPr2 | mismatches |
| HPV51 | TTATTGGCTCCACCGT | 0 |
| HPV18 | A------T-A--TAAG | 7 |

TABLE 4d

| | Hybridized to | | Signal | target/probe | Signal/ | | |
|---|---|---|---|---|---|---|---|
| Probe | target | Hybr. proc. | (MFI) | (%) | noise | Remark | Exp |
| 18MLPr7T40 | MPF HPV18 | Thermo Cycler | 1082 | 100 | 144 | Specific | ID148 |
| 18MLPr7T40 | MPF HPV51 | Thermo Cycler | 6 | 1 | 1 | Negative | ID148 |
| 51MLPr2T40 | MPF HPV51 | Thermo Cycler | 1410 | 100 | 123 | Specific | ID148 |
| 51MLPr2T40 | MPF HPV18 | Thermo Cycler | 20 | 1 | 1 | Negative | ID148 |
| 18MLPr7T40 | MPF HPV18 | Thermo Mixer | 2154 | 100 | 287 | Specific | ID148 |
| 18MLPr7T40 | MPF HPV51 | Thermo Mixer | 6 | 0 | 1 | Negative | ID148 |
| 51MLPr2T40 | MPF HPV51 | Thermo Mixer | 2725 | 100 | 210 | Specific | ID148 |
| 51MLPr2T40 | MPF HPV18 | Thermo Mixer | 25 | 1 | 2 | Negative | ID148 |

### Tables Example 5

TABLE 5a
51SLPr2 = SPF₁₀ probe 51 version 2,
C₁₂ = a stretch of 12 carbon atoms

| Name | Probe composition |
|---|---|
| 51SLPr2 | NH₂—C₁₂-CTATTTGCTGGAACAATC |

TABLE 5b
Identical nucleotides are indicated by a “-”.

| | Alignment with probe | Number of |
|---|---|---|
| Target | 51SLPr2 | mismatches |
| HPV 51 | CTATTTGCTGGAACAATC | 0 |
| HPV 33 | T------T---GG----- | 4 |
| HPV 16 | -------T---GGT---- | 4 |

TABLE 5c

| | | PE inc. temp. | Signal | target/ | Signal/ | | |
|---|---|---|---|---|---|---|---|
| Probe | Hybridized to target | (° C.) | (MFI) | probe (%) | noise | Remark | Exp |
| 51SLPr2 | SPF₁₀ HPV51 | 50 | 3681 | 100 | 194 | Specific | ID44 |
| 51SLPr2 | SPF₁₀ HPV33 | 50 | 345 | 9 | 18 | Cross react | ID44 |
| 51SLPr2 | SPF₁₀ HPV16 | 50 | 30 | 1 | 2 | Negative | ID44 |
| 51SLPr2 | SPF₁₀ HPV51 | RT | 3074 | 100 | 615 | Specific | ID43 |
| 51SLPr2 | SPF₁₀ HPV33 | RT | 259 | 8 | 52 | Cross react | ID43 |
| 51SLPr2 | SPF₁₀ HPV16 | RT | 5 | <1 | 1 | Negative | ID43 |

TABLE 5d

| | | Wash temp. | Signal | target/ | Signal/ | | |
|---|---|---|---|---|---|---|---|
| Probe | Hybridized to target | (° C.) | (MFI) | probe (%) | noise | Remark | Exp |
| 51SLPr2 | SPF₁₀ HPV51 | 50 | 2433 | 100 | 187 | Specific | ID90 |
| 51SLPr2 | SPF₁₀ HPV33 | 50 | 423 | 16 | 33 | Cross react | ID90 |
| 51SLPr2 | SPF₁₀ HPV16 | 50 | 8 | <1 | 1 | Negative | ID90 |
| 51SLPr2 | SPF₁₀ HPV51 | RT | 2777 | 100 | 179 | Specific | ID90 |
| 51SLPr2 | SPF₁₀ HPV33 | RT | 374 | 13 | 24 | Cross react | ID90 |
| 51SLPr2 | SPF₁₀ HPV16 | RT | 10 | <1 | 1 | Negative | ID90 |

### Tables Example 7

TABLE 7a
51SLPr2 = SPF₁₀ probe 51 version 2,
C₁₂ = a stretch of 12 carbon atoms

| Name | Probe composition |
|---|---|
| 51SLPr2 | NH₂—C₁₂-CTATTTGCTGGAACAATC |

TABLE 7b
Identical nucleotides are indicated by a “-”.

| | Alignment with probe | Number of |
|---|---|---|
| Target | 51SLPr2 | mismatches |
| HPV 51 | CTATTTGCTGGAACAATC | 0 |
| HPV 31 | T------T---GG----- | 4 |

TABLE 7c

| | | Storage 4° C. | Signal | target/ | Signal/ | | |
|---|---|---|---|---|---|---|---|
| Probe | Hybridized to target | (hrs) | (MFI) | probe (%) | noise | Remark | Exp |
| 51SLPr2 | SPF₁₀ HPV51 | 0 | 1573 | 100 | 51 | Specific | ID110 |
| 51SLPr2 | SPF₁₀ HPV31 | 0 | 30 | 2 | 1 | Negative | ID110 |
| 51SLPr2 | SPF₁₀ HPV51 | 4 | 1611 | 100 | 59 | Specific | ID111 |
| 51SLPr2 | SPF₁₀ HPV31 | 4 | 28 | 2 | 1 | Negative | ID111 |
| 51SLPr2 | SPF₁₀ HPV51 | 24 | 1783 | 100 | 60 | Specific | ID113 |
| 51SLPr2 | SPF₁₀ HPV31 | 24 | 34 | 2 | 1 | Negative | ID113 |
| 51SLPr2 | SPF₁₀ HPV51 | 96 | 1707 | 100 | 52 | Specific | ID114 |
| 51SLPr2 | SPF₁₀ HPV31 | 96 | 33 | 2 | 1 | Negative | ID114 |

### Tables Example 8

TABLE 8a
51SLPr2 = SPF₁₀ probe 51 version 2,
C₁₂ = a stretch of 12 carbon atoms,
C₁₈ = a stretch of 18 carbon atoms

| Name | Probe composition |
|---|---|
| 51SLPr2 | NH₂—C₁₂-CTATTTGCTGGAACAATC |
| 51SLPr2C₁₈ | NH₂—C₁₈-CTATTTGCTGGAACAATC |
| 33SLPr21 | NH₂—C₁₂-GGGCAATCAGGTATT |
| 33SLPr21C₁₈ | NH₂—C₁₈-GGGCAATCAGGTATT |

TABLE 8b
Identical nucleotides are indicated by a “-”.

| | Alignment with probe | Number of |
|---|---|---|
| Target | 51SLPr2 | mismatches |
| HPV 51 | CTATTTGCTGGAACAATC | 0 |
| HPV 33 | T------T---GG----- | 4 |

TABLE 8c
Identical nucleotides are indicated by a “-”.

| | Alignment with probe | Number of |
|---|---|---|
| Target | 33SLPr21 | mismatches |
| HPV 33 | GGGCAATCAGGTATT | 0 |
| HPV 51 | -AA---------C-T-- | 4 |

TABLE 8d

| | | | | Signal/ | | |
|---|---|---|---|---|---|---|
| Probe | Hybridized to target | Signal (MFI) | target/probe (%) | noise | Remark | Exp |
| 51SLPr2 | SPF₁₀ HPV51 | 4291 | 100 | 172 | Specific | ID64 |
| 51SLPr2 | SPF₁₀ HPV33 | 358 | 8 | 14 | Cross reaction | ″ |
| 51SLPr2C₁₈ | SPF₁₀ HPV51 | 3515 | 100 | 216 | Specific | ID67 |
| 51SLPr2C₁₈ | SPF₁₀ HPV33 | 16 | 0 | 1 | Negative | ″ |
| 33SLPr21 | SPF₁₀ HPV33 | 429 | 100 | 48 | Specific | ID77 |
| 33SLPr21 | SPF₁₀ HPV51 | 52 | 12 | 6 | Cross reaction | ″ |
| 33SLPr21C₁₈ | SPF₁₀ HPV33 | 429 | 100 | 61 | Specific | ″ |
| 33SLPr21C₁₈ | SPF₁₀ HPV51 | 4 | 1 | 1 | Negative | ″ |

### Tables Example 9

TABLE 9a
51SLPr2 = SPF₁₀ probe 51 version 2,
C₁₂ = a stretch of 12 carbon atoms,
(T)₄₀ = a stretch of 40 Thymine nucleotides

| Name | Probe composition |
|---|---|
| 51SLPr2 | NH₂—C₁₂-CTATTTGCTGGAACAATC |
| 51SLPr2T10 | NH₂—C₁₂-(T)₁₀-CTATTTGCTGGAACAATC |
| 51SLPr2T20 | NH₂—C₁₂-(T)₂₀-CTATTTGCTGGAACAATC |
| 51SLPr2T30 | NH₂—C₁₂-(T)₃₀-CTATTTGCTGGAACAATC |
| 51SLPr2T40 | NH₂—C₁₂-(T)₄₀-CTATTTGCTGGAACAATC |

TABLE 9b
52MLPr2 = MPF probe 52 version 2,
C₁₂ = a stretch of 12 carbon atoms,
(T)₄₀ = a stretch of 40 Thymine nucleotides

| Name | Probe composition |
|---|---|
| 52MLPr2 | NH₂—C₁₂-CCGTACTGGTTACAACGA |
| 52MLPr2T20 | NH₂—C₁₂-(T)₂₀-CCGTACTGGTTACAACGA |
| 52MLPr2T30 | NH₂—C₁₂-(T)₃₀-CCGTACTGGTTACAACGA |
| 52MLPr2T40 | NH₂—C₁₂-(T)₄₀-CCGTACTGGTTACAACGA |

TABLE 9c
Identical nucleotides are indicated by a “-”.

| | Alignment with probe | Number of |
|---|---|---|
| Target | 51SLPr2 | mismatches |
| HPV 51 | CTATTTGCTGGAACAATC | 0 |
| HPV 33 | T------T---GG----- | 4 |

TABLE 9d
Identical nucleotides are indicated by a “-”.

| | Alignment with probe | Number of |
|---|---|---|
| Target | 52MLPr2 | mismatches |
| HPV 52 | CCGTACTGGTTACAACGA | 0 |
| HPV 16 | --T--T------------ | 2 |

TABLE 9e

| | | | target/probe | | | |
|---|---|---|---|---|---|---|
| Probe | Hybridized to target | Signal (MFI) | (%) | Signal/noise | Remark | Exp |
| 51SLPr2 | SPF₁₀ HPV51 | 4291 | 100 | 172 | Specific | ID64 |
| 51SLPr2 | SPF₁₀ HPV33 | 358 | 8 | 14 | Cross reaction | ID64 |
| 51SLPr2T10 | SPF₁₀ HPV51 | 4688 | 100 | 122 | Specific | ID64 |
| 51SLPr2T10 | SPF₁₀ HPV33 | 34 | 1 | 1 | Negative | ID64 |
| 51SLPr2T20 | SPF₁₀ HPV51 | 8712 | 100 | 387 | Specific | ID64 |
| 51SLPr2T20 | SPF₁₀ HPV33 | 32 | 0 | 1 | Negative | ID64 |
| 51SLPr2T30 | SPF₁₀ HPV51 | 8077 | 100 | 414 | Specific | ID64 |
| 51SLPr2T30 | SPF₁₀ HPV33 | 30 | 0 | 1 | Negative | ID64 |
| 51SLPr2T40 | SPF₁₀ HPV51 | 7356 | 100 | 320 | Specific | ID64 |
| 51SLPr2T40 | SPF₁₀ HPV33 | 32 | 0 | 1 | Negative | ID64 |

TABLE 9f

| | | | target/probe | | | |
|---|---|---|---|---|---|---|
| Probe | Hybridized to target | Signal (MFI) | (%) | Signal/noise | Remark | Exp |
| 51MLPr2 | MPF HPV52 | 423 | 100 | 13 | Specific | ID69 |
| 51MLPr2 | MPF HPV16 | 32 | 8 | 1 | Cross reaction | ID69 |
| 51MLPr2T20 | MPF HPV52 | 1233 | 100 | 95 | Specific | ID69 |
| 51MLPr2T20 | MPF HPV16 | 11 | 1 | 1 | Negative | ID69 |
| 51MLPr2T30 | MPF HPV52 | 1250 | 100 | 139 | Specific | ID69 |
| 51MLPr2T30 | MPF HPV16 | 8 | 1 | 1 | Negative | ID69 |
| 51MLPr2T40 | MPF HPV52 | 1510 | 100 | 126 | Specific | ID69 |
| 51MLPr2T40 | MPF HPV16 | 9 | 1 | 1 | Negative | ID69 |

### Tables Example 10

TABLE 10a
18MLPr7 = MPF probe 18 version 7,
C₁₂ = a stretch of 12 carbon atoms,
(T)₄₀ = a stretch of 40 Thymine nucleotides,
(TTG)₁₃ = a stretch of 13 Thymine-
Thymine-Guanine nucleotide triplets
(39 nucleotides total)

| Name | Probe composition |
|---|---|
| 18MLPr7T40 | NH₂—C₁₂-(T)₄₀-TTACATAAGGCACAGG |
| 18MLPr7(TTG)₁₃ | NH₂—C₁₂-(TTG)₁₃-TTACATAAGGCACAGG |

TABLE 10b

| | | | target/probe | | | |
|---|---|---|---|---|---|---|
| Probe | Hybridized to target | Signal (MFI) | (%) | Signal/noise | Remark | Exp |
| 18MLPr7T40 | MPF HPV18 | 2001 | 100 | 13 | Specific | ID169 |
| 18MLPr7T40 | nc8 | 1104 | 54 | 7 | Cross reaction | ID169 |
| 18MLPr7T40 | DNA− | 2 | 0 | 0 | Negative | ID169 |
| 18MLPr7(TTG)₁₃ | MPF HPV18 | 2390 | 100 | 199 | Specific | ID169 |
| 18MLPr7(TTG)₁₃ | nc8 | 23 | 1 | 2 | Negative | ID169 |
| 18MLPr7(TTG)₁₃ | DNA− | 2 | 0 | 0 | Negative | ID169 |

nc8 = negative control 8 showing cross reaction with all probes in a LiPA assay,
DNA− = negative control

### Tables Example 11

TABLE 11a
18MLPr7 = MPF probe 18 version 7,
C₁₂ = a stretch of 12 carbon atoms,
(T)₄₀ = a stretch of 40 Thymine nucleotides,
N5 = 5′-end amino linker, N3 = 3′-end
amino linker

| Name | Probe composition |
|---|---|
| 18MLPr7T40N5 | NH₂—C₁₂-(T)₄₀-TTACATAAGGCACAGG |
| 18MLPr7T40N3 | TTACATAAGGCACAGG-(T)₄₀-C₁₂—NH₂ |
| 45MLPr8T40N5 | NH₂—C₁₂-(T)₄₀-CCAGGGCCATAACAAG |
| 45MLPr8T40N3 | CCAGGGCCATAACAAG-(T)₄₀-C₁₂—NH₂ |

TABLE 11b

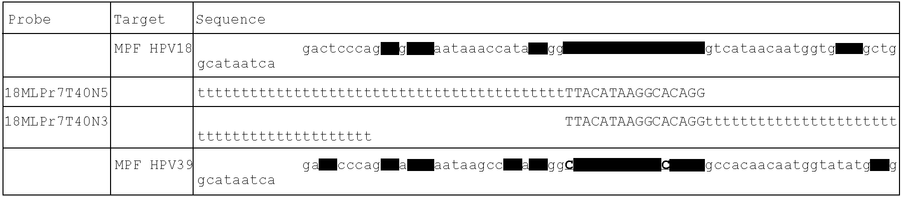

18MLPr7 = MPF probe 18 version 7, N5 = 5'-end amino linker, N3 = 3'-end amino linker, gray boxed sequence = target nucleotides that may bind to Thymine spacer (lower case) and probe sequence (upper case), bold & underlined = mismatch with probe sequence.

TABLE 11c

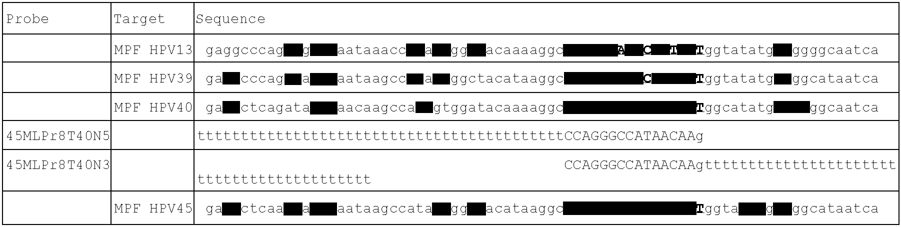

45MLPr8 = MPF probe 45 version 8, N5 = 5'-end amino linker, N3 = 3'-end amino linker, gray boxed sequence = target nucleotides that may bind to Thymine spacer (lower case) and probe sequence (upper case), bold & underlined = mismatch with probe sequence.

TABLE 11d

| | | | target/probe | | | |
|---|---|---|---|---|---|---|
| Probe | Hybridized to target | Signal (MFI) | (%) | Signal/noise | Remark | Exp |
| 18MLPr7T40N5 | MPF HPV18 | 1146 | 100 | 85 | Specific | ID141 |
| 18MLPr7T40N5 | MPF HPV39 | 518 | 45 | 38 | Cross reaction | ID141 |
| 18MLPr7T40N3 | MPF HPV18 | 694 | 100 | 139 | Specific | ID141 |
| 18MLPr7T40N3 | MPF HPV39 | 12 | 2 | 2 | Negative | ID141 |
| 45MLPr8T40N5 | MPF HPV13 | 611 | 38 | 51 | Cross reaction | ID141 |
| 45MLPr8T40N5 | MPF HPV39 | 284 | 18 | 24 | Cross reaction | ID141 |
| 45MLPr8T40N5 | MPF HPV40 | 1021 | 64 | 85 | Cross reaction | ID141 |
| 45MLPr8T40N5 | MPF HPV45 | 1600 | 100 | 133 | Specific | ID141 |
| 45MLPr8T40N3 | MPF HPV13 | 47 | 8 | 8 | Cross reaction | ID141 |
| 45MLPr8T40N3 | MPF HPV39 | 17 | 3 | 3 | Negative | ID141 |
| 45MLPr8T40N3 | MPF HPV40 | 116 | 19 | 19 | Cross reaction | ID141 |
| 45MLPr8T40N3 | MPF HPV45 | 615 | 100 | 103 | Specific | ID141 |

TABLES 12a and b

| MFI | | | % target/probe | | |
|---|---|---|---|---|---|
| | Bead/ | Bead/ | | Bead/ | Bead/ |
| | probe | probe | | probe | probe |
| Target | A1 | A2 | Target | A1 | A2 |
| a | 988 | 4399 | a | 100 | 100 |
| b | 13 | 14 | b | 1 | 0 |
| c | 19 | 19.5 | c | 2 | 0 |
| d | 5 | 13 | d | 1 | 0 |
| e | 3 | 4 | e | 0 | 0 |
| f | 11 | 6 | f | 1 | 0 |
| g | 14 | 9 | g | 1 | 0 |
| h | 3 | 3 | h | 0 | 0 |

% target/probe:
A1, a = 988/988 * 100 = 100%;
A1, c = 19/988 * 100 = 2%

TABLES 13a and b

| MFI | | | Signal/noise | | |
|---|---|---|---|---|---|
| | Bead/ | Bead/ | | Bead/ | Bead/ |
| | probe | probe | | probe | probe |
| Target | A1 | A2 | Target | A1 | A2 |
| A | 988 | 4399 | a | 82 | 400 |
| B | 13 | 14 | b | 1 | 1 |
| C | 19 | 19.5 | c | 2 | 2 |
| D | 5 | 13 | d | 0 | 1 |
| E | 3 | 4 | e | 0 | 0 |
| F | 11 | 6 | f | 1 | 1 |
| G | 14 | 9 | g | 1 | 1 |
| H | 3 | 3 | h | 0 | 0 |
| Median | 12 | 11 | | | |

Signal/noise:
A1, a = 988/12 (= median (988, 13, 19, 5, 3, 11, 14, 3)) = 82;
A1, c = 19/12 (median (988, 13, 19, 5, 3, 11, 14, 3)) = 2.

1. A probe suitable for coupling with a particulate support, the probe comprising:
a) a coupling group which permits coupling of the probe to the surface of the particulate support;
b) a spacer; and
c) a target-specific oligonucleotide probe sequence,
wherein the spacer comprises:
i) an oligonucleotide spacer of at least 15 nucleotides between the target specific probe sequence and the support coupling group; and optionally
ii) a carbon spacer of between 3 and 50 carbon units between the target specific probe sequence and the support coupling group.

2. A probe according to claim 1 wherein the spacer comprises both a carbon spacer and an oligonucleotide spacer.

3. A probe according to claim 1 wherein the spacer is an oligonucleotide spacer without a carbon spacer and is from 25-150 nucleotides.

4. A probe according to claim 1 wherein the spacer is selected so as not to hybridise to the target sequence or a flanking region of the target.

5. A probe according to claim 1 wherein the oligonucleotide spacer comprises a homopolymer or a heteropolymer.

6. A probe according to claim 5 wherein the oligonucleotide spacer comprises poly T oligonucleotide or TTG repeats.

7. A probe according to claim 1 wherein the target specific probe sequence is specific for a human papillomavirus target sequence.

8. A probe according to claim 1 coupled to a particulate support.

9. A probe according to claim 9 wherein the support is a bead.

10. A probe according to claim 8 wherein the support is selected from glass or polystyrene.

11. A set of probes according to claim 1, comprising at least two different target specific probe sequences coupled to different particulate supports which are distinguishable from one another.

12. A set of probes according to claim 11 wherein the different particulate supports are labelled with different fluorescent molecules.

13. A set of from 2 to 1000 different target specific probes, each probe comprising:
a) a coupling group which permits coupling of the probe to a particulate support;
b) a spacer; and
c) a target-specific oligonucleotide probe sequence,
wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group, which oligonucleotide spacer does not hybridise to the target sequence or a flanking region of the target.

14. A spacer suitable for attachment to a target specific probe sequence comprising a carbon spacer of between 13 and 50 units and an oligonucleotide of at least 15 nucleotides.

15. A kit comprising a probe which probe comprises:
a) a coupling group which permits coupling of the probe to a surface of a particulate support;
b) a spacer; and
c) a target-specific oligonucleotide probe sequence,
wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group;
and a particulate support such as polystyrene beads.

16. A kit comprising a probe which probe comprises:
a) a coupling group which permits coupling of the probe to a surface of a particulate support;
b) a spacer; and
c) a target-specific oligonucleotide probe sequence,
wherein the spacer comprises one or both of:
i) a carbon spacer of between 13 and 50 carbon units between the target specific probe sequence and the support coupling group; and
ii) an oligonucleotide spacer of at least 15 nucleotides between the target specific probe sequence and the support coupling group;
and instructions for coupling to a particulate support such as polystyrene beads.

17. A kit according to claim 15 which comprises a set of two or more different target specific probe sequences for coupling to different particulate supports which are distinguishable from one another.

18. A method for the detection of any interaction between a probe according to claim 1 and a target nucleic acid, the method comprising the steps of:
i) denaturation of any double stranded target polynucleic acid present in a sample;
ii) hybridisation of the denatured target with probe under conditions that allow specific hybridization between probe and target to occur;
iii) (optionally, stringent washing)
iv) addition of, and incubation with, reporter molecule to allow detection of probe-target binding;
v) (optionally, washing); and
vi) detection of probe-target binding
wherein the method comprises maintenance of the hybridization temperature from step ii) until the end of step iv).

19. A method according to claim 18 wherein the probe is coupled to a particulate support such as a bead.

20. A method according to claim 18 wherein 2 or more different probes are used simultaneously.

21. A method according to claim 18 wherein the target-specific probe sequence length of different target-specific probes is not identical.

22. A method according to claim 18 wherein hybridization between probe and target is carried out in an ionic environment.
